(19) United States
(12) Patent Application Publication
(10) Pub. No.: US 2010/0041966 A1
(43) Pub. Date: Feb. 18, 2010

(54) SLEEP ASSISTANT SYSTEM AND METHOD THEREOF

(75) Inventors: SHIH-CHUNG KANG, Taipei (TW); RAYLEIGH PING-YING CHIANG, Taipei City (TW); PETER LIU, Taipei (TW)

Correspondence Address: MICHAEL W. TAYLOR, P.O. BOX 3791, ORLANDO, FL, 32802-3791, US

(73) Assignee: National Taiwan University, Taipei (TW)

(21) Appl. No.: 12/538,966

(22) Filed: Aug. 11, 2009

(30) Foreign Application Priority Data
Aug. 14, 2008 (TW) 97131037

(51) Int. Cl. A61B 5/00

(52) U.S. Cl. 600/301

(57) ABSTRACT

A sleep assistant method for a sleeper in an environment is provided. The method includes the steps of monitoring a bio-condition of the sleeper to collect a bioinformation of the sleeper; performing a sleep coach mode, which analyzes the bioinformation and provides a sleep knowledge to the sleeper based on the analyzed results; and performing a sleep environment adjusting mode, which adjusts the environment based on the bioinformation.

### FIELD OF THE INVENTION

The present invention relates to a sleep assistant system for a sleeper, especially to a sleep assistant system for a sleeper to build a good sleep environment and to provide a sleep coach program to the sleeper.

### BACKGROUND OF THE INVENTION

About one third of the human lifetime is spent on sleeping. Thus, the sleep is quite important. A good life quality is usually built up with the good sleep quality. To improve the basic of the life quality should start at the improvement of the sleep quality. Unfortunately, according to the research findings, about 11.7% of Americans (i.e. about 32 million people) suffer from the problem of insomnolence or sleeplessness. The patients are widely distributed in different ages, sexes, races and social levels. The insomnolence affects the life quality largely. When the sleep is unbalanced, the physical and psychological conditions will be largely influenced, and even the family, job and social relationships are impacted as well. Therefore, it is necessary to solve the sleep problem and to do the research for building up high quality sleep environments.

Generally speaking, people easily fall asleep in more comfortable, safe, and familiar environment. One of the methods to solve the sleep problem is to build an “optimum sleep environment”. However, currently the developments of building the optimum sleep environment still focus on the development and the integration of the monitoring apparatus in the medical engineering to monitor various action indexes of the sleeper, e.g. brain wave, respiration, snoring sound, muscle tension, oximetric concentration, etc., which can be used by doctor to diagnose various sleep disorders and to evaluate the improving conditions before and after the treatment. Undoubtedly, the current society imminently requires an apparatus and method for providing appropriate sleep knowledge to the user and controlling and building good sleep environments.

### SUMMARY OF THE INVENTION

The present invention provides a sleep assistant system and a sleep assistant method for a sleeper to improve the sleep quality and to solve the sleep problem of the sleeper.

In accordance with one aspect of the present invention, a sleep assistant method for a sleeper in an environment is provided. The method comprises monitoring a bio-condition of the sleeper to collect a bioinformation of the sleeper; performing a sleep coach mode, which analyzes the bioinformation and provides a sleep knowledge to the sleeper based on the analyzed results; and performing a sleep environment adjusting mode, which adjusts the environment based on the bioinformation.

Preferably, the bioinformation includes at least one selected from a group consisting of a heart beat, a body temperature, a blood pressure, a skin conductivity and a respiration rate.

Preferably, the sleep knowledge includes at least one selected from a group consisting of a direction from a doctor, a prescription by a doctor, a sleep medical knowledge, a medical treatment knowledge, a pre-sleep action and a sleep skill.

In accordance with another aspect of the present invention, a sleep environment adjusting system for a sleeper in an environment having an environment parameter is provided. The system comprises a sensor sensing the sleeper to obtain a bioinformation; an environment adjusting device disposed in the environment; and an electronic processor electrically connected with the sensor and the environment adjusting device, and controlling the environment adjusting device based on the bioinformation.

Preferably, the sensor is disposed on the sleeper, and the electronic processor has a sleep environment adjusting mode controlling the environment adjusting device to adjust the environmental parameter based on the bioinformation and a preference of the sleeper.

Preferably, the environmental parameter comprises at least one selected from a group consisting of a visual effect, an acoustic volume, a temperature, a humidity and an air condition.

Preferably, the environment adjusting device comprises at least one selected from a group consisting of an air conditioner, an illuminating device, an audio device, a wake-up device and a combination thereof.

Preferably, the electronic processor is electrically connected with the sensor and the environment adjusting device via one of a wireless and a wired connections.

Preferably, the sleep environment adjusting mode is based on a fuzzy algorithm program.

In accordance with a further aspect of the present invention, a sleep coach apparatus for a sleeper is provided. The apparatus comprises a sensor sensing the sleeper to obtain a bioinformation; and an electronic processor electrically connected with the sensor, and providing a sleep knowledge to the sleeper based on the bioinformation.

Preferably, the sensor is a biosensor, and the sleeper is one of a person ready to sleep and a person falling asleep.

Preferably, the bioinformation comprises at least one selected from a group consisting of a heart beat, a body temperature, a blood pressure, a skin conductivity and a respiration rate.

Preferably, the electronic processor is one selected from a group consisting of a personal computer, a notebook computer and a server, and is electrically connected with the sensor via one of a wireless and a wired connections.

Preferably, the apparatus further comprises a display device electrically connected with the electronic processor.

Preferably, the electronic processor has a sleep coach mode analyzing the bioinformation and providing the sleep knowledge to the sleeper via the display device.

Preferably, the sleep coach mode comprises an intellectual algorithm program, which is designed based on at least one of a direction from a doctor and a prescription by a doctor.

Preferably, the display device comprises one selected from a group consisting of a cathode ray tube display, a liquid crystal display, a plasma display, a touch panel display and a projector.

Preferably, the sleep knowledge comprises at least one selected from a group consisting of a direction from a doctor, a prescription by a doctor, a sleep medical knowledge, a medical treatment knowledge, a pre-sleep action and a sleep skill.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is the schematic diagram showing the sleep assistant system of the present invention;

FIG. 2 is the schematic diagram showing the module configurations of the sleep assistant apparatus of the present invention;

FIG. 3 is the schematic diagram showing the flow chart for performing the sleep assistant method of the present invention.

FIG. 4 is the schematic diagram showing the concepts of the sleep assistant method of the present invention.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

The present invention proposes an apparatus and a method for building good sleep environments. The relevant factors influencing the sleep are quite diversified. Both the conditions of the sleeper and the environment must be considered simultaneously, and make it complicated to build the appropriate sleep environment, since what each person recognizes and feels is different. Therefore, the optimum sleep environment would be different as the requirements of the sleep environment for each person are different. All the life pace, physical environments and psychological factors and so on would influence the quality of the sleep. The influence factors related to the life pace include the busy and tense modern life and the job time difference. The influence factors related to the physical environments include light, sound, temperature, air quality, bedding and pillow, etc. The influence factors related to the psychological factors include thinking and the psychological response to the stimulation of the environments. Usually all the above factors would influence the sleep quality of a person, and thus are taken into account and integrated into the sleep assistant system of the present invention to build up the optimum sleep environments for a sleeper or a sleeper-centered bedroom.

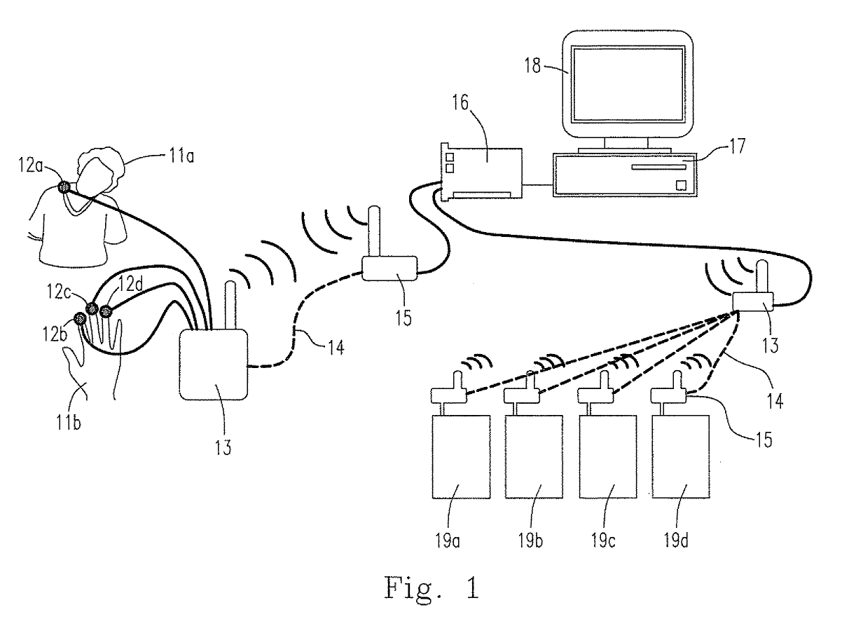

Please refer to FIG. 1, which shows the sleep assistant system of the present invention. The FIG. 1 shows a body 11a, a hand 11b of a sleeper, sensors 12a, 12b, 12c and 12d, front transceiver 13, data transmission cord 14, rear transceiver 15, data acquisition device 16, personal computer (PC) 17, display device 18, environment adjusting devices 19a, 19b, 19c and 19d, and the connection relationships among the above devices or elements. In this embodiment, the sleeper means a person ready to sleep or a person falling asleep, and the sleep means pre-sleep or during sleeping.

In this embodiment, the PC-based data acquisition (DAQ) configuration is adopted to collect and process various bioinformation data of the sleeper. However, the present invention is not limited to the PC-based data acquisition structure. Instead of the PC-based DAQ, any data acquisition configuration able to collect and process various bioinformation data of the sleeper can also be integrated into the sleep assistant system of the present invention to reach the same effect of the present invention.

The sensors 12a, 12b, 12c and 12d in this embodiment can be biosensors, and can be disposed in body 11a, hand 11b or other part of the body of the sleeper to collect/monitor/measure/sense the raw data of various bio-action indexes, including heart beat, blood pressure, skin conductivity, respiration condition, etc. The monitoring of these bioinformations usually proceeds prior to sleep or during the sleep. These bioinformations can be used to check the sleep quality of the sleeper. The biosensors in the market or in the research can be integrated into the sleep assistant system of the present invention to acquire the monitored bioinformation of a sleeper prior to sleep or during the sleep. These monitored bioinformations can be extended to more knowledge in the sleep medicine field to further understand the sleep behavior of the human beings.

The signals of the bioinformations can be collected by the sensors 12a, 12b, 12c and 12d, and transmitted to front transceiver 13. These signals can be digital or analog signals depending on whether the inputs/outputs (I/O) of the sensors 12a, 12b, 12c and 12d are digital or analog. The front transceiver 13 and rear transceiver 15 can be wireless transceivers or wired transceivers. The front transceiver 13 transmits the signals of the bioinformations from the sensors 12a, 12b, 12c and 12d to the rear transceiver 15, and receives the signals or commands from the rear transceiver 15. When the wireless communication is adopted for the front and rear transceivers, various wireless communication techniques can be integrated into the sleep assistant system of the present invention, such as IEE 802.11a (5 GHz), IEEE 802.11b/g (2.4 GHz), IEEE 802.11n (5 and/or 2.4 GHz), bluetooth (2.4 GHz), and Worldwide Interoperability for Microwave Access (WiMAX, 2.3, 2.5 and 3.5 GHz). Alternately, the front transceiver 13 can be connected with rear transceiver 15 by data transmission cord 14.

After the sensors 12a, 12b, 12c and 12d sense the bioinformations of the sleeper, the data acquisition device 16 acquires the raw data transmitted by rear transceiver 15, and transmits these data to PC 17 for the subsequent data processing or computation. In the PC-based configuration, data acquisition device 16 is a DAQ card. If the analog I/O configuration is adopted for the sensors, data acquisition device 16 can be an AD converter DAQ card to convert the acquired analog signals into the digital signals.

To sum up, in an embodiment of the present invention, the sensors 12a, 12b, 12c and 12d collect the raw data of the bioinformations, and data acquisition device 16 acquires and transmits these raw data to PC 17 for the subsequent data processing. In an embodiment, data acquisition device 16 is directly electrically connected with the sensors 12a, 12b, 12c and 12d to acquire the signals without passing through front transceiver 13 and rear transceiver 15.

Furthermore, in the sleep assistant system of the present invention, PC 17 is electrically connected with the display device 18. The PC 17 contains the software of the sleep coach mode and the sleep environment adjusting mode. In an embodiment of the present invention, the sleep coach mode and the sleep environment adjusting mode are set up in the LabView program in the PC-based configuration. After PC 17 receives the signals from data acquisition device 16, the sleep coach mode and the sleep environment adjusting mode can obtain various bioinformations from data acquisition device 16.

The sleep coach mode installed in PC 17 is an intellectual algorithm program, is designed based on the direction from a doctor or a prescription by a doctor, and can analyze the monitored bioinformations to obtain the indexes of the sleep quality, e.g. the relative percentage of each periods during the sleep, arousal index, cyclic alternating pattern (CAP), etc. Accordingly, it can be determined what kind of sleep knowledge is going to be provided to the sleeper. This sleep knowledge can include the direction from the doctor, the prescription by the doctor, the sleep medical knowledge, medical treatment knowledge, proper pre-sleep actions, sleep skills, etc. After the sleep coach mode further analyzes the monitored bioinformations, the medical suggestions or prescriptions will be shown on the display device 18 of the PC 17 as the visual interface, which is a communication means between the sleep coach mode and the sleeper, and can be designed as an interactive way to response to the medical suggestions. After the sleep knowledge is transmitted to the sleeper via this clear and easy-understanding way, the physical and psychological conditions of the sleeper can be effectively changed, and the sleep quality of the sleeper can be gradually improved.

That is, after PC 17 receives the signals from data acquisition device 16, the sleep coach mode analyzes these signals of the bioinformations, and then display device 18 provides the appropriate and helpful sleep knowledge to the sleeper. Here the display device 18 can be a cathode ray tube (CRT) display, a liquid crystal display (LCD), a plasma display, a touch panel display or a projector.

The sleep environment adjusting mode installed in PC 17 can be a program based on a fuzzy algorithm, and can adjust the environment where the sleeper is located based on the monitored bioinformations. The sleep environment adjusting mode receives the bioinformation from the sleeper, then determines the relative important levels of each bioinformation, and then decides how to adjust various sleep quality influence factors, including the sound, light, room temperature, air condition, etc. The sleep environment adjusting mode can be further integrated with the architectural technique to control the visual and audio effects so as to build up the good sleep environment. Then the environment factors can be specifically tailored for the individual sleeper to build up the appropriate sleep environment to improve the sleep quality and to effectively assist the treatment for the sleep disorder.

The environment adjusting devices 19a, 19b, 19c and 19d can be the air conditioner, illumination device, audio device and wake-up device. The air conditioner can be the window air conditioner, separated air conditioner or central air conditioner. The illumination device can be a fluorescent lamp, a desk lamp, a stand lamp or a bed lamp. The wake-up device can be an alarm clock.

When PC 17 receives the signals from data acquisition device 16, the sleep environment adjusting mode analyzes these signal of the bioinformations, and the analyzed results are transmitted to rear transceiver 15 via data acquisition device 16 and front transceiver 13 to control the environment adjusting device 19a, 19b, 19c and 19d so as to adjust the environment factors. In an embodiment of the present invention, the analyzed results from the data acquisition device 16 can be used to directly control the environment adjusting devices 19a, 19b, 19c and 19d without pass front transceiver 13 and rear transceiver 15.

Basically, a sleep coach apparatus can include the sensors 12a, 12b, 12c and 12d, front transceiver 13, cord 14, rear transceiver 15, data acquisition device 16, PC 17 and display device 18. The sleep environment adjusting system can include the sensors 12a, 12b, 12c and 12d, front transceiver 13, cord 14, rear transceiver 15, data acquisition device 16, PC 17, display device 18 and environment adjusting devices 19a, 19b, 19c and 19d.

The above sleep assistant system can be set up in any space, including the bedrooms, hotels, dormitories or hospitals.

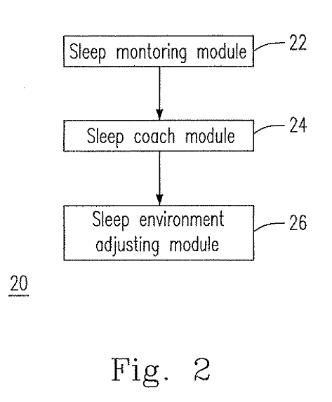

The sleep assistant system of the present invention can be further divided into several modules for facilitating the modulized implementations according to the different functions for each stage. Please refer to FIG. 2, which shows the module configurations of the sleep assistant apparatus of the present invention. The sleep assistant apparatus 20 in FIG. 2 includes the sleeper monitoring module 22, sleep coach module 24 and sleep environment adjusting module 26.

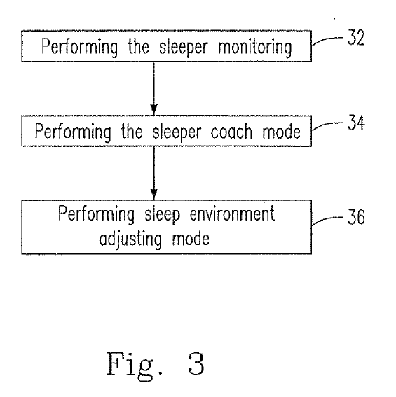

To sum up the above mentioned concepts of the present invention, a sleep assistant method can be obtained. Please refer to FIG. 3, which shows the flow chart for performing the sleep assistant method of the present invention. The method in FIG. 3 includes the steps of performing the sleeper monitoring 32, performing the sleeper coach mode 34, and performing the sleep environment adjusting mode 36.

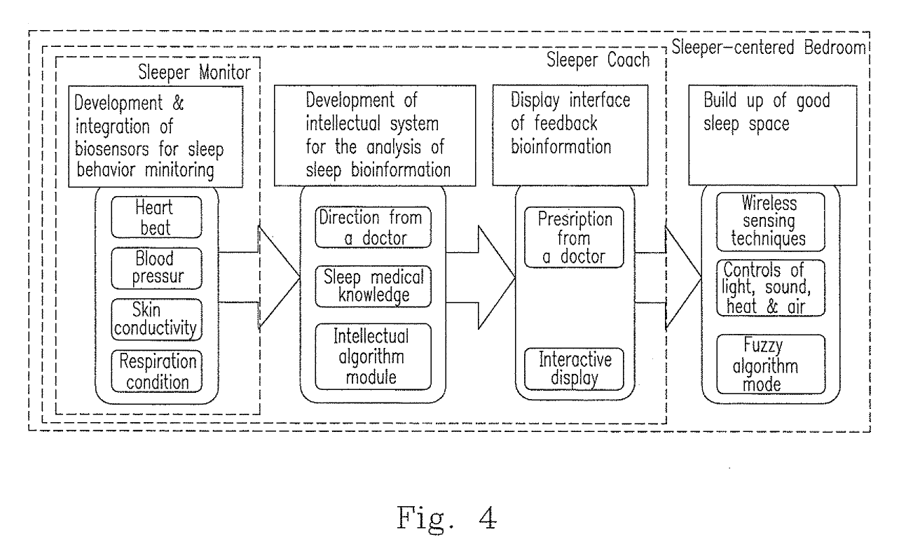

From the above, the clear concepts shown in FIG. 4 can be obtained. Please refer to FIG. 4, which shows the concepts of the sleep assistant method of the present invention.

In conclusion, the sleep assistant apparatus and the sleep assistant method of the present invention are based on the economic and portable sleep monitoring device. The biosensors are developed and integrated into the monitoring of the sleep behaviors. The conditions of the sleeper are sensed by the biosensors with the portability or easy installation so as to build up the optimum sleep environment. The sleep monitoring devices are used to monitor various bio-action indexes during the sleep, e.g. heart beat, body temperature, skin conductivity, respiration rate, etc. For the patients with the sleep disorder, these bio-action indexes can be used for the doctor's diagnosis and the comparison of the conditions before and after the treatments. Further cooperating with the other sensing devices in the intellectual space, an integrated caring network can be built. The monitored indexes before and during the sleep can be further analyzed as the reference for the subsequent diagnosis.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A sleep assistant method for a sleeper in an environment, comprising:
monitoring a bio-condition of the sleeper to collect a bioinformation of the sleeper;
performing a sleep coach mode, which analyzes the bioinformation and provides a sleep knowledge to the sleeper based on the analyzed results; and
performing a sleep environment adjusting mode, which adjusts the environment based on the bioinformation.

2. A method according to claim 1, wherein:
the bioinformation includes at least one selected from a group consisting of a heart beat, a body temperature, a blood pressure, a skin conductivity and a respiration rate; and
the sleep knowledge includes at least one selected from a group consisting of a direction from a doctor, a prescription by a doctor, a sleep medical knowledge, a medical treatment knowledge, a pre-sleep action and a sleep skill.

3. A sleep environment adjusting system for a sleeper in an environment having an environment parameter, comprising:
a sensor sensing the sleeper to obtain a bioinformation;
an environment adjusting device disposed in the environment; and
an electronic processor electrically connected with the sensor and the environment adjusting device, and controlling the environment adjusting device based on the bioinformation.

4. A system according to claim 3, wherein the sensor is disposed on the sleeper, and the electronic processor has a sleep environment adjusting mode controlling the environment adjusting device to adjust the environmental parameter based on the bioinformation and a preference of the sleeper.

5. A system according to claim 3, wherein the sensor is a biosensor, and the sleeper is one of a person ready to sleep and a person falling asleep.

6. A system according to claim 3, wherein the bioinformation comprises at least one selected from a group consisting of a heart beat, a body temperature, a blood pressure, a skin conductivity and a respiration rate.

7. A system according to claim 3, wherein the environmental parameter comprises at least one selected from a group consisting of a visual effect, an acoustic volume, a temperature, a humidity and an air condition.

8. A system according to claim 3, wherein the environment adjusting device comprises at least one selected from a group consisting of an air conditioner, an illuminating device, an audio device, a wake-up device and a combination thereof.

9. A system according to claim 3, wherein the electronic processor is one selected from a group consisting of a personal computer, a notebook computer and a server, and is electrically connected with the sensor and the environment adjusting device via one of a wireless and a wired connections.

10. A system according to claim 3, wherein the sleep environment adjusting mode is based on a fuzzy algorithm program.

11. A sleep coach apparatus for a sleeper, comprising:
a sensor sensing the sleeper to obtain a bioinformation; and
an electronic processor electrically connected with the sensor, and providing a sleep knowledge to the sleeper based on the bioinformation.

12. An apparatus according to claim 11, wherein the sensor is a biosensor, and the sleeper is one of a person ready to sleep and a person falling asleep.

13. An apparatus according to claim 11, wherein the bioinformation comprises at least one selected from a group consisting of a heart beat, a body temperature, a blood pressure, a skin conductivity and a respiration rate.

14. An apparatus according to claim 11, wherein the electronic processor is one selected from a group consisting of a personal computer, a notebook computer and a server, and is electrically connected with the sensor via one of a wireless and a wired connections.

15. An apparatus according to claim 11, further comprising a display device electrically connected with the electronic processor.

16. An apparatus according to claim 15, wherein the electronic processor has a sleep coach mode analyzing the bioinformation and providing the sleep knowledge to the sleeper via the display device.

17. An apparatus according to claim 16, wherein the sleep coach mode comprises an intellectual algorithm program, which is designed based on at least one of a direction from a doctor and a prescription by a doctor.

18. An apparatus according to claim 15, wherein the display device comprises one selected from a group consisting of a cathode ray tube display, a liquid crystal display, a plasma display, a touch panel display and a projector.

19. An apparatus according to claim 11, wherein the sensor is disposed on the sleeper.

20. An apparatus according to claim 11, wherein the sleep knowledge comprises at least one selected from a group consisting of a direction from a doctor, a prescription by a doctor, a sleep medical knowledge, a medical treatment knowledge, a pre-sleep action and a sleep skill.
